(19) United States
(12) Patent Application Publication
(10) Pub. No.: US 2008/0161884 A1
(43) Pub. Date: Jul. 3, 2008

(54) Method and apparatus for treating or preventing a medical condition

(76) Inventors: Mark Chandler, Southern Pines, NC (US); Andrea Moretti, London (GB); Richard Nagle, London (GB); Christopher John Rainey, Devon (GB); John Peter Southgate, Devon (GB)

Correspondence Address: DICKSTEIN SHAPIRO LLP, 1825 EYE STREET NW, Washington, DC, 20006-5403, US

(21) Appl. No.: 11/878,855

(22) Filed: Jul. 27, 2007

Related U.S. Application Data
(63) Continuation-in-part of application No. 11/019,237, filed on Dec. 23, 2004.

(51) Int. Cl. A61N 1/32; A61N 2/04; A61N 7/00; A61F 5/00

(52) U.S. Cl. 607/50; 600/13; 601/2; 607/148; 602/2

(57) ABSTRACT

A method of treating a wound or preventing injury to a region susceptible to injury comprising the steps of placing a pair of electrodes placed spaced apart in the region of a wound and applying a predetermined sequence of current waveforms across the electrodes. The sequence of current waveforms comprises a first waveform comprising a series of current pulses having an amplitude in a range of from 80 to 300 μA, having a frequency in a range from 0.5 to 1.5 pulses per second and a pulse width in a range from 333 to 1000 ms, a second waveform comprising a series of current pulses having an amplitude in a range of from 20 to 60 μA, having a frequency in a range from 2 to 4 pulses per second and a pulse width in a range from 125 to 250 ms and a third waveform comprising a series of current pulses having an amplitude in a range of from 250 to 640 μA, having a frequency in a range of from 80 to 120 pulses per second and a pulse width in a range from 4 to 6 ms.

### CROSS REFERENCE TO RELATED APPLICATIONS

The present application is a continuation-in-part of U.S. patent application Ser. No. 11/019,237, filed on Dec. 23, 2004 (published as US2006142817A1).

Related patent applications U.S. Ser. No. 11/138,358 (published as US2006173523A1), and U.S. Ser. No. 11/802,201, International applications PCT/GB2006/00315 (published as WO2006082384), PCT/GB2007/00317 (published as WO 2007/088348) and PCT/GB2007/01842 (published as WO 2008/003920) are incorporated in their entirety herein by reference thereto. UK patent application GBO701129.9 is incorporated by reference.

### FIELD OF THE INVENTION

The present invention generally relates to a method and apparatus for treating or preventing a medical condition. In particular, the invention relates to a method and apparatus for treating an area of injured tissue such as a wound, or for preventing injury to tissue susceptible to injury, such as the formation of a wound, involving application of electrical signals to the region of injured or susceptible tissue.

### BACKGROUND OF THE INVENTION

Healing and prevention of medical conditions requires effective treatment. For example, chronic wounds such as venous ulcers which form and do not heal, represent a serious problem to sufferers and healthcare providers. The prevalence of active venous ulcers in the adult population is high and their treatment is very costly to healthcare services. A venous ulcer is an area of damage to the skin that can occur when the veins and muscles in the lower legs are weak and cannot efficiently pump the blood back to the heart as a result of damaged valves. Gravity then causes the blood to pool in the lower legs. The pooling blood eventually leaks out of the veins and into the surrounding tissue causing the tissue to swell, which then leads to wounds and ulcers. Such wounds may be extremely painful and grow to a considerable size.

Applying increased external pressure to the legs with compression bandaging or graduated compression hosiery, for example, has been used to assist in the healing of venous leg ulcers and gravitational eczema. The increased pressure forces the blood back into the veins creating an improved blood flow thereby allowing the ulcers to heal. Compression bandages or graduated compression hosiery apply pressure to the leg, with greater pressure near the ankle and reduced pressure higher up. This forces the blood to keep circulating away from the lower leg and can, by reducing venous dilation, partially restore correct venous valve action whilst applied.

Studies have shown that the process of healing, growth and regeneration in living tissue is brought about by the flow of endogenous electrical current. It has been suggested that the application of external microcurrents to injured tissue can assist the body's natural healing process by augmenting the flow of current through the injured tissue. The application of electrical signals to injured tissue as a form of therapy is known as electrotherapy and has been described in various publications.

U.S. Pat. No. 4,982,742 describes a method and apparatus for facilitating the healing of soft tissue wounds involving the application of a single bi-phase microcurrent waveform to a selected area of tissue. The waveform is characterised by a frequency ranging from 10 to 50 Hz and an amplitude ranging between 100 and 1000 μA. The waveform is delivered by a disposable bandage containing an integrated circuit and power source.

Similarly the method described in U.S. Pat. No. 6,393,326 uses one waveform throughout treatment. The electrical treatment signal disclosed in this document is characterised by a bipolar voltage waveform at a frequency of between 2 Hz and 10 Hz. This method is particularly adapted to the treatment of bedsores that are known to have substantially zero electrical activity.

EP367320 also relates to a system for the treatment of wounds by electric stimulation. The document discloses a waveform generator adapted to generate either a direct current signal or a pulsed signal comprising pulses with a pulse width of less than 1 ms. It further discloses that optimal pulse width is about 0.1 ms. The DC current application is believed to produce wound healing and the pulse signals when applied directly into the wounds are said to produce a pain-relief effect.

There is a recognised need for an effective method for preventing the formation of and for promoting the healing of chronic wounds such as venous ulcers. It would be particularly advantageous to have a method of electrotherapy tailored to the prevention and/or healing of venous ulcers.

### SUMMARY OF THE INVENTION

The present invention provides a method of and an apparatus for treating injured tissue or preventing injury to tissue susceptible to injury. Examples of injured tissue include wounds, ulcers, bruising, inflammation, blistering etc. Tissue susceptible to these types of injury includes tissue susceptible to the formation of wounds, ulcers, bruising, inflammation, blistering etc.

In one aspect of the invention the method includes placing a plurality of electrodes spaced apart in the region of said tissue, placing a covering exerting pressure on the region of the wound to improve blood flow in the region of the wound, and applying a sequence of predetermined electrical waveforms across a pair of electrodes placed spaced apart in the region of the said tissue. The sequence of waveforms includes a first waveform comprising a series of current pulses having an amplitude of between 80 and 300 μA, having a frequency of between 0.5 and 1.5 pulses per second and a pulse width of between 333 and 1000 ms, a second waveform comprising a series of current pulses having an amplitude of between 20 and 60 μA, a frequency of between 2 and 4 pulses per second and a pulse width of between 125 and 250 ms, and a third waveform comprising a series of current pulses having an amplitude of between 250 and 640 μA, having a frequency of between 80 and 120 pulses per second and a pulse width of between 4 and 6 ms. The application of waveforms with these ranges of parameters increases efficacy in the healing of wounds. Thus in accordance with this aspect of the invention, the benefits of conventional pressure bandages and an electrotherapy treatment specific for the treatment of wounds caused by blood pooling such as venous ulcers are achieved.

The polarity of the electrodes may be reversed approximately every 5 to 15 seconds during application of the first, second and/or third waveform.

In one embodiment, the first waveform comprises a series of current pulses having an amplitude of substantially 100 μA, a frequency of substantially 1 pulse per second and a pulse width of substantially 500 ms, the second waveform comprises a series of current pulses having a amplitude of substantially 40 μA, a frequency of substantially 3 pulses per second and a pulse width of substantially 166 ms, the third waveform comprises a series of current pulses having an amplitude of substantially 320 μA, a frequency of substantially 100 pulses per second and a pulse width of substantially 5 ms.

In an embodiment, the electrodes are positioned in contact with skin peripheral to the said tissue. The skin peripheral to the injured tissue is likely to be unbroken.

In an embodiment, each electrode of a pair of electrodes is positioned on opposite sides of the injured or susceptible tissue to one another so that the current passes through regenerative tissue under the said tissue.

In an embodiment, each electrode is placed approximately 1 cm from an outer boundary of the said tissue.

In an embodiment, each waveform is generated over a period of time ranging from 5 to 40 minutes.

In an embodiment, the first waveform is generated over a period of time ranging from 5 to 10 minutes, the second waveform is generated over a period of time ranging from 25 to 45 minutes and the third waveform is generated over a period of time ranging from 20 to 35 minutes.

In one embodiment, the pulses are substantially rectangular. This encompasses pulses that are functionally rectangular or square.

In one embodiment, the sequence of waveforms is repeated thus providing treatment over a longer period of time if required.

In a second aspect of the invention a method of treating injured tissue or tissue susceptible to injury involves placing a plurality of electrodes in contact with skin in a region peripheral to said tissue, placing a compression covering over the electrodes and the region of the wound to improve blood flow in the region of the said tissue and applying an electrical current to pass from at least one electrode to another electrode of the plurality of electrodes. This aspect of the present invention provides simultaneous conventional pressure bandage treatment and electrotherapy treatment to provide an improved method of treating injured tissue such as wounds and an improved method of preventative treatment for tissue susceptible to the formation of injuries such as wounds.

In one embodiment, the end of each electrode extends beyond the outermost edges of said tissue, for example a wound, so that the entire surface of said tissue is positioned between two electrodes.

In one embodiment, each electrode extends beyond the outermost edges of said tissue, for example a wound, by approximately 1.0 to 1.5 cm.

In a third aspect of the invention, a method of treating a wound includes placing a plurality of electrodes in contact with skin in a region peripheral to the wound and applying a sequence of specific current waveforms between the electrodes. The sequence of specific waveforms includes a first waveform comprising a series of current pulses having an amplitude in a range of from 80 to 300 μA, having a frequency in a range from 0.5 to 1.5 pulses per second and a pulse width in a range from 333 to 1000 ms, a second waveform comprising a series of current pulses having an amplitude in a range of from 20 to 60 μA, having a frequency in a range from 2 to 4 pulses per second and a pulse width in a range from 125 to 250 ms and a third waveform comprising a series of current pulses having an amplitude in a range of from 250 to 640 μA, having a frequency in a range of from 80 to 120 pulses per second and a pulse width in a range from 4 to 6 ms. The application of this sequence of waveforms optimises wound healing. This aspect of the invention provides a new and improved electrotherapy treatment wherein the electrodes are not placed on the wound but in the periwound, thus avoiding deteriously interfering with the wound healing process and allowing for the administering of electrical current therapy through the regenerative tissue under the wound.

The apparatus according to a fourth aspect of the invention includes a waveform generator adapted to generate a predetermined sequence of waveforms comprising three waveforms and output connectors for connection to one or more pair of electrodes for applying the sequence of waveforms under the wound.

In one embodiment, the apparatus includes a polarity switch for reversing the polarity of the electrodes.

In an embodiment, the waveform generator is pre-programmed with one or more programs for generating one of said waveforms or a pre-determined sequence of said waveforms.

In one embodiment, the apparatus includes a user interface for selecting one of said waveforms or a predetermined sequence of said waveforms.

In one embodiment, the apparatus includes a second waveform generator for supplying a predetermined sequence of waveforms to a second pair of electrodes.

According to a fifth aspect of the invention there is provided a wound treatment arrangement for treating a wound in the skin of a patient. The wound treatment arrangement comprises a compression dressing arrangement applied to a limb of the patient and applying pressure thereto; a plurality of electrodes for applying electrical signals to the skin of a patient wherein said electrodes are arranged on said skin adjacent said wound under said compression dressing arrangement; and an electrical generator for generating electrical current to pass from one of the plurality of electrodes to another of the plurality of electrodes.

A sixth aspect of the invention provides a kit of parts for treating a wound in the skin of a patient, the kit comprising a compression dressing arrangement for application to the limb of the patient to apply pressure thereto; a plurality of electrodes for applying electrical signals to the skin of the patient; and an electrical generator for generating electrical current to pass from one of the plurality of electrodes to the other of the plurality of electrodes.

A seventh aspect of the invention provides method of treating a medical condition on the surface of or under the surface of a region of tissue, the method comprising: positioning a plurality of electrodes spaced apart in the region of tissue; placing a compression covering over the plurality of electrodes and the region of said tissue to apply pressure in the region of said tissue; and applying a sequence of electrical waveforms between at least two of the plurality of electrodes, the sequence of electrical waveforms comprising: a first waveform comprising a series of current pulses having an amplitude in a range of from 80 to 300 μA, having a frequency in a range from 0.5 to 1.5 pulses per second and a pulse width in a range from 333 to 1000 ms; a second waveform comprising a series of current pulses having an amplitude in a range of from 20 to 60 μA, having a frequency in a range from 2 to 4 pulses per second and a pulse width in a range from 125 to 250 ms; and a third waveform comprising a series of current pulses having an amplitude in a range of from 250 to 640 μA, having a frequency in a range of from 80 to 120 pulses per second and a pulse width in a range from 4 to 6 ms.

Examples of such medical conditions include injured and defective veins or valves located under the surface of the skin.

The method of treating injured tissue or preventing injury to tissue susceptible to injury or medical conditions according to the invention has the advantage that it is capable of working in combination with other treatment methods promoting the healing of wounds such as venous leg ulcers such as the application of compression bandaging to the area of treatment. In addition, it is beneficial to have a method of treatment consisting of different treatment phases. It is also advantageous to have a method for promoting the healing of wounds or preventing the formation of wounds that is non-invasive, that is easy to apply and that is capable of being used on a long term basis.

### BRIEF DESCRIPTION OF THE DRAWINGS

Embodiments of the invention will now be described with reference to the accompanying drawings, in which:—

FIG. 1 is a perspective view of a device for generating electrical signals according to a first embodiment of the present invention;

FIG. 2 is a schematic diagram showing one channel of a device for generating electrical signals according to the first embodiment of the present invention;

FIG. 3 is a schematic diagram of a waveform generator according to the first embodiment of the present invention;

FIG. 4 is a schematic diagram of the area of treatment showing the disposition of electrode pads according to the first embodiment of the present invention;

FIG. 5 is a simplified illustration of a method of treating a venous leg ulcer according to the first embodiment of the invention.

FIG. 6A is a graphical illustration of a first waveform generated by the waveform generator according to the first embodiment of the present invention;

FIG. 6B is a graphical illustration of a second waveform generated by the waveform generator according to the first embodiment of the present invention;

FIG. 6C is a graphical illustration of a third waveform generated by the waveform generator according to the first embodiment of the present invention;

FIG. 7A is a schematic view of a second embodiment of apparatus for treating an area of injured tissue according to the invention;

FIG. 7B illustrates the apparatus of FIG. 7a fitted on the leg of a patient;

FIG. 8A is cross-sectional view of a third embodiment of an electrode arrangement for applying electrical signals to a region of treatment;

FIG. 8B is a planar view of the electrode arrangement of FIG. 8a;

FIG. 9A-9C are schematic views of an apparatus for treating a wound in accordance with a fourth embodiment of the invention;

FIG. 10 is a schematic view of an apparatus for treating a wound according to a fifth embodiment of the invention;

FIG. 11 is a schematic view of a treatment device according to a sixth embodiment of the invention;

FIGS. 12A and 12B are top views of electrode designs according to a seventh and eighth embodiment of the invention respectively;

FIGS. 12C and 12D are corresponding bottom views of an electrode designs according to the seventh and eighth embodiment of the invention respectively; and

FIGS. 13C to 13E illustrate various placements of the electrodes of the seventh embodiment of the present invention.

### DETAILED DESCRIPTION OF THE EMBODIMENTS

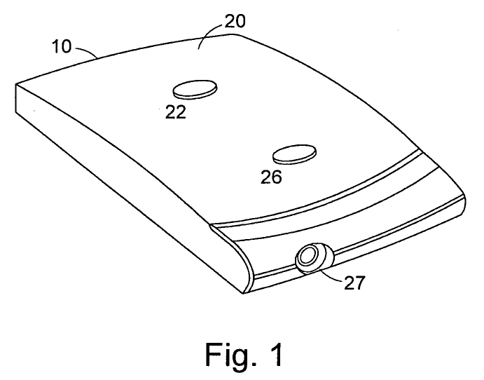

FIG. 1 is a perspective view of an electrotherapy device 10 for applying electrical signals to an area of tissue according to a first embodiment of the present invention. The electrotherapy device 10 comprises a housing 20, a waveform generator channel having an electrode port 27, an input switch 26 and an on/off switch 22. The input switch 26 and the on/off switch 22 may be of the push button type. The housing 20 encloses the waveform generator channel 30.

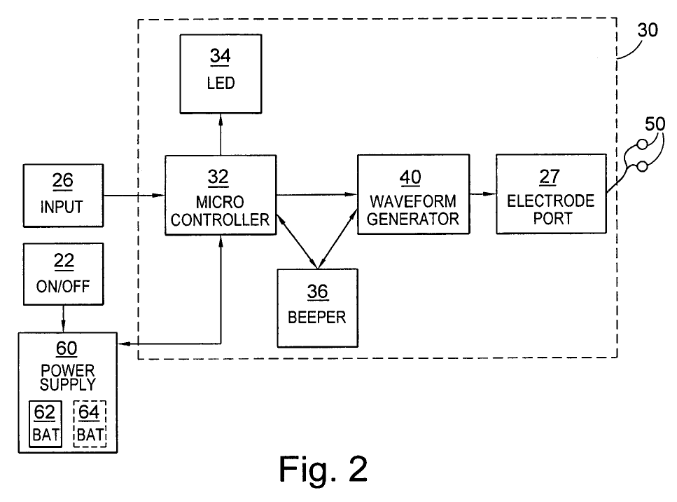

FIG. 2 is a schematic diagram of the device 10 showing the waveform generator channel 30. The channel 30 includes an electrode port 27, a microcontroller 32, a waveform generator 40, an LED unit 34 and a beeper 36. The channel 30 is connected to the input switch 26, a power supply 60 and a pair of electrodes 50. The electrodes 50 may be of any type known in the art of electrotherapy. Power supply 60 supplies the microcontroller 32 and the rest of the channel 30 with power and is controlled by on/off switch 22. In this embodiment the power supply supplies power to the channel 30 by means of a battery. Turning on the device 10 via the on/off switch 22 activates the power supply 60, which in turn controls the on/off status of the battery 62 (and 64 where appropriate). In this embodiment the power supply 60 converts the battery voltage to a supply logic level of five volts.

The microcontroller 32 controls and/or monitors voltage, input switch 26, LED unit 34, beeper 36 and the waveform generator 40. The waveform generator 40 receives signals from the microcontroller 32, and accordingly generates an appropriate current waveform, and supplies the waveform to the electrode port 27. The electrodes 50 transfer the waveform from the electrode port 27 to the tissue to be treated. The input switch 26 and the on/off switch 22 are resistor multiplexed into an analog port of the microcontroller 32.

The status of the device 10 is indicated by LEDs of the LED unit 34, controlled by microcontroller 32. Beeper 36 is activated when the device 10 detects high resistance between the individual electrodes of the electrode pair 50, indicating that the electrodes 50 are not making proper contact with the portion of the body to be treated. Such a situation is called a pad open condition. Beeper 36 is also activated when other error conditions such as a low battery voltage are detected.

The device 10 is activated via on/off switch 22. Once energized, the microcontroller checks switch 26. Switch 26 is used to start a pre-programmed three-stage waveform treatment program. The microcontroller 32 sends appropriate signals to the waveform generator 40 based on the pre-programmed three-stage waveform treatment program to cause the appropriate signals to be sent to the electrodes 50. The microcontroller also instructs the LED unit 34 and the beeper 36 to indicate the status of device 10. The device 10 automatically cycles through the three-stage treatment program when switch 26 is pressed, and automatically switches off when the cycle of treatment stages has finished. During the treatment program a ticking noise is emitted by the beeper 36 to indicate that the program is running. The beeper 36 emits a series of beeps at the end of the treatment program to indicate that the program has finished.

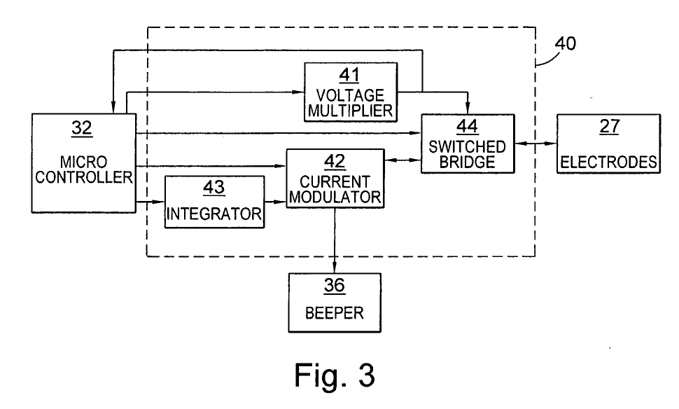

The waveform generator 40 is shown in more detail in FIG. 3. The waveform generator comprises a voltage multiplier 41, a current modulator 42, an integrator 43, and a switched bridge 44. The combined elements of the waveform generator 40 take power from the power supply 60 and generate a current waveform under control of the microcontroller 32.

The voltage multiplier supplies a voltage pumped signal to the switched bridge 44. In this embodiment, the voltage multiplier multiplies the battery voltage by 6. The voltage multiplier includes a voltage feedback loop with the microcontroller 32. The feedback voltage is fed to an ADC and software reduces the drive frequency to reduce the output voltage as required.

The switched bridge 44 supplies the generated current waveform to the electrode port 27. In this embodiment, the switched bridge 44 comprises four opto-isolators in a bridge configuration. In addition to the voltage pumped signal from the voltage multiplier 41, the switched bridge receives a polarity control signal from the microcontroller 32 and a current modulation signal from the current modulator 42. The integrator 42 processes the waveform signals received from the microcontroller 32 resulting in ramp, sine and square wave outputs as required. These outputs are sent to the current modulator 42. The current modulator 42 controls the output current level under direction of the microcontroller 32. A software-switchable sense resistor controls the current range. The current modulator 42 receives signals from the integrator 43 and also receives current control signals from the microcontroller 32.

The microcontroller 32 supplies various signals to various portions of the waveform generator 40 so as to generate appropriate current waveforms. For example, the microcontroller 32 supplies a modulated square wave signal to the voltage multiplier 41, an output polarity setting to the switched bridge 44, a pulse width modulated synthesized waveform to the integrator 43 and a current level selection signal to the current modulator 42. The waveform parameters are stored in an EPROM and cannot be modified by the user. The microcontroller 32 can be considered as being functionally part of the waveform generator. The waveform generator 40 supplies electrical signals to electrodes 50 via the electrode port 27.

In alternative embodiments the device may include two or more channels for simultaneously or alternately transmitting electrical signals to two or more electrode ports. A second channel may communicate with the first channel through an opto-isolator.

In further embodiments of the invention, the device 10 may include other forms of display for displaying data representative of the device status. It will be appreciated that in some embodiments, the microcontroller and the waveform generator may constitute one unit.

In yet further embodiments the device 10 may be programmed with two or more waveform treatment programs for generating a predetermined waveform or a predetermined sequence of waveforms. The device may further include a user interface to select between different waveform treatment programs. In some embodiments the device may be programmable by a health care provider.

In an alternative embodiment of the invention device 10 may be replaced with another form of electrotherapy device for generating the electrical waveforms such as that described in U.S. application U.S. Ser. No. 11/138,358 (published as US2006173523A1) and International application PCT/GB2006/00315 (published as WO2006082384) both entitled “Electrode Arrangement for applying electrical signals to the skin of an animal” and in the name of Wound Solutions Ltd, incorporated in their entirety herein with specific reference thereto.

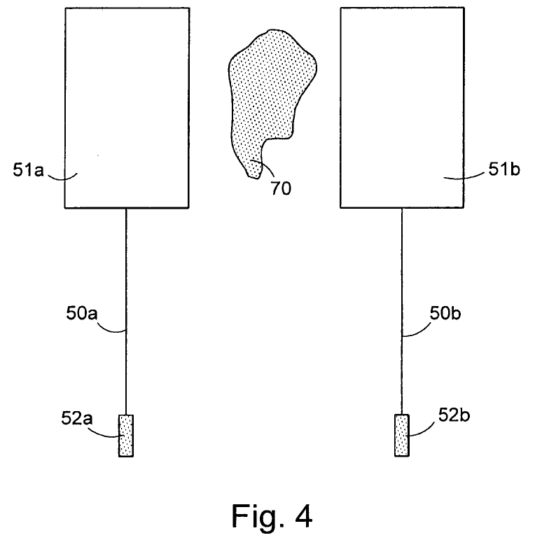

A method of treatment in accordance with an embodiment of the present invention will now be described with reference to FIGS. 4 to 6.

The method of the present invention includes steps of arranging electrodes 50 around the area of body to be treated, covering the electrodes and the area of body to be treated with a compression bandage, providing a first electrotherapy waveform during a first treatment stage, providing a second electrotherapy waveform during a second treatment stage and providing a third electrotherapy waveform during a third treatment stage.

The electrical waveforms are administered to an area of a body via the pair of electrodes made up of electrode pads 51a and 51b which are placed on the surface of the skin on opposite sides of a wound 70 substantially parallel to the major axis of the wound as shown in FIG. 4. The electrode pads adhere to the skin of the patient and disperse current evenly across the surface. The electrode pads may be of any type known in electrotherapy and may be available in different sizes. The inner edge of the electrode pads are placed approximately 1 cm from the outer edges of the wound. Ends of the electrode pads are arranged to extend approximately 1.5 cm beyond the outer edges of the wound in both directions substantially parallel to the major axis of the wound. The electrode pads are connected to a pair of electrode leads 50a and 50b which are terminated by a pair of connectors 52a and 52b respectively, at one end for connection to the electrode port 27 of the device 10. Since the electrodes are placed outside the wound there is no need to remove and reapply any wound dressing and the wound is not irritated by contact with the electrode pads.

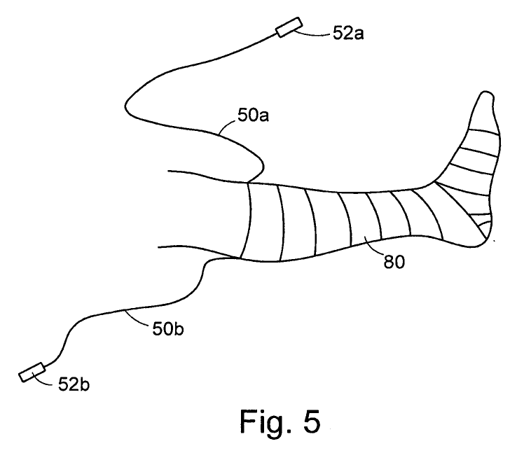

The method according to this embodiment is particularly suited to the treatment of venous leg ulcers. The electrodes pads are placed around the wound on the leg and the leg is wrapped in a compression bandage 80 as shown in FIG. 5 Electrode leads 50a and 50b protrude from the compression bandaging 80 for connection to the electrotherapy device 10. The leads are between 5 m and 50 m in length. In this embodiment of the invention, a four layer compression bandaging system is used. The compression bandage applies pressure to the leg with greater pressure near the ankle and reduced pressure higher up. This forces the blood to keep circulating away from the lower leg and reduces blood pooling in the lower area. The improved blood flow aids in the healing of the wound. Since the electrode connectors protrude from the bandaging they are easily accessible for connection to the electrotherapy device and there is no need to wrap and unwrap the compression bandage. Since there is no need to unwrap the bandage each time treatment is administered this is practical for both the patient receiving treatment and the carer administering the treatment. Furthermore, the electrotherapy treatment works in combination with compression treatment providing benefits of the two treatments simultaneously to the patient.

Although FIG. 5 shows the electrode leads protruding from the top of the compression bandaging, in alternative embodiments the electrode leads may protrude from the side of the bandaging in the region of the wound so that the electrode leads do not have to be pressed along the length of the leg. In some embodiments the waveform generator may remain permanently on the leg throughout the life of the bandage and may be retained by outer layers of the bandage.

In a further embodiment the electrode leads may be manufactured from a flat material such as conductive fabric and can be laid on the surface of the skin under the bandage up to the top of said bandage.

In a further alternative embodiment each electrode may have a pin protruding outwards which pierces the bandage as it is applied, and to which electrical connection is subsequently made.

In a still further alternative embodiment the waveform generator may be placed under some or all of the layers of the bandaging, and may be attached to one or more of the electrodes. In a still further alternative embodiment parts of the waveform generator may be placed under some or all of the layers of the bandaging, and may be connected wirelessly with the remainder of the waveform generator, said wireless connection being by inductive coupling or electromagnetic coupling.

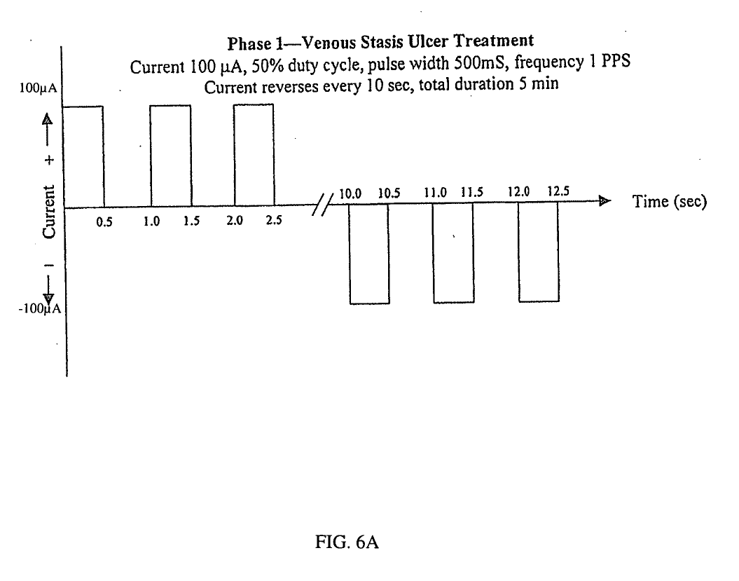

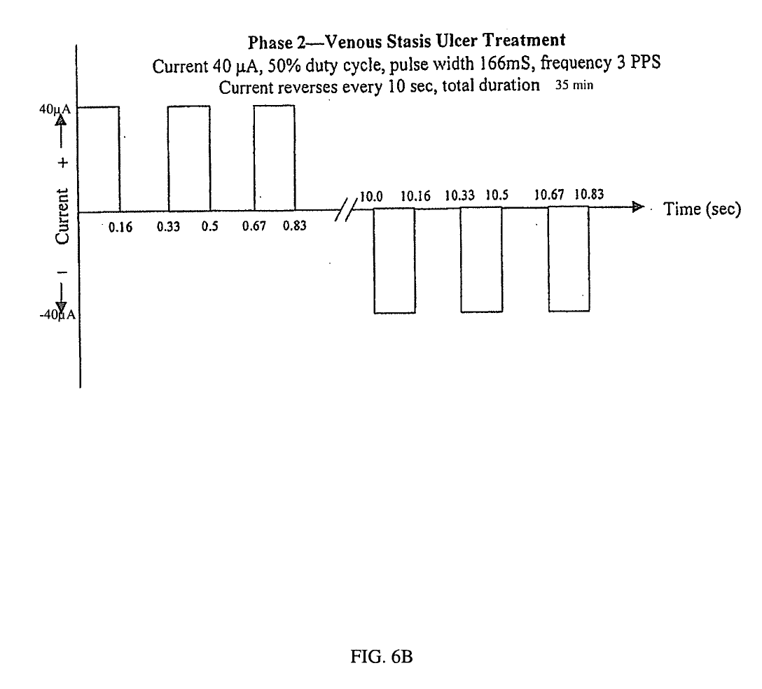

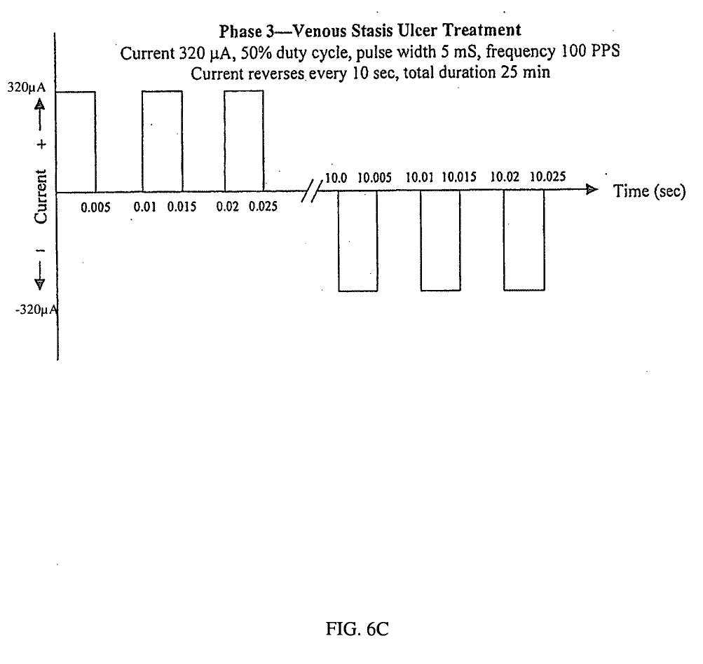

In a first stage of treatment the waveform illustrated in FIG. 6A is applied to the treatment area. The first treatment stage is particularly suited to reducing the resistance of the injured tissue. It has been proposed that injured tissue has a higher electrical resistance than healthy tissue such that the flow of natural electrical current through an injured section of the body is lower than the flow through normal surrounding tissue. The decreased electrical flow through the injured tissue decreases the cellular capacitance. Consequently, healing of the injured tissue is impaired. It has been further proposed that reducing the resistance of injured tissue and allowing the body's natural bio-electricity to enter the area would aid the healing process or reduce pain. To facilitate a change in tissue resistance the electrodes are provided with a waveform comprising a series of current pulses with an amplitude of 100 μA, having a frequency of 1 pulse per second (pps) and a pulse width of 500 mS. The pulses are substantially square and are characterised by a rapid rise to a current level, a hold at that current level, followed by a rapid return to near zero current. The polarity of the electrodes is reversed at periodic intervals of approximately 10 seconds. This stage of treatment lasts for 5 minutes.

In a second stage of treatment the waveform illustrated in FIG. 6B is applied to the treatment area. The second stage of treatment is particularly suited to healing injured tissue by providing a current that mimics the body's natural current. To facilitate healing of the injured tissue the electrodes are provided with a waveform comprising a series of pulses with an amplitude of 40 μA, having a frequency of 3 pps and a pulse width of 166 ms. The polarity of the electrodes is reversed at periodic intervals of approximately 10 seconds. The second stage of treatment lasts for 35 minutes.

In a third stage of treatment the waveform illustrated in FIG. 6C is applied to the treatment area. The third stage of treatment is particularly suited to promoting blood vessel regeneration (angiogenesis). To facilitate blood vessel regeneration in injured tissue the electrodes are provided with a waveform comprising a series of pulses with an amplitude of 320 μA, having a frequency of 100 pps and a pulse width of 5 ms. The third treatment stage lasts for 25 minutes.

The three treatment stages are automatically executed sequentially. The second treatment stage follows the first treatment stage and the third treatment stage follows the second treatment stage. Since the three treatment stages are executed sequentially there is no need for further user interaction beyond starting the treatment program. The patient is free to relax and read a book or watch television while receiving treatment. The treatment can be administered by the patient himself in the comfort of his own home without the need to go to hospital. An advancement whereby the device is an ambulatory device attached to the leg and requires zero interference by the patient as the device delivers the treatment waveforms automatically for the required number of times per day.

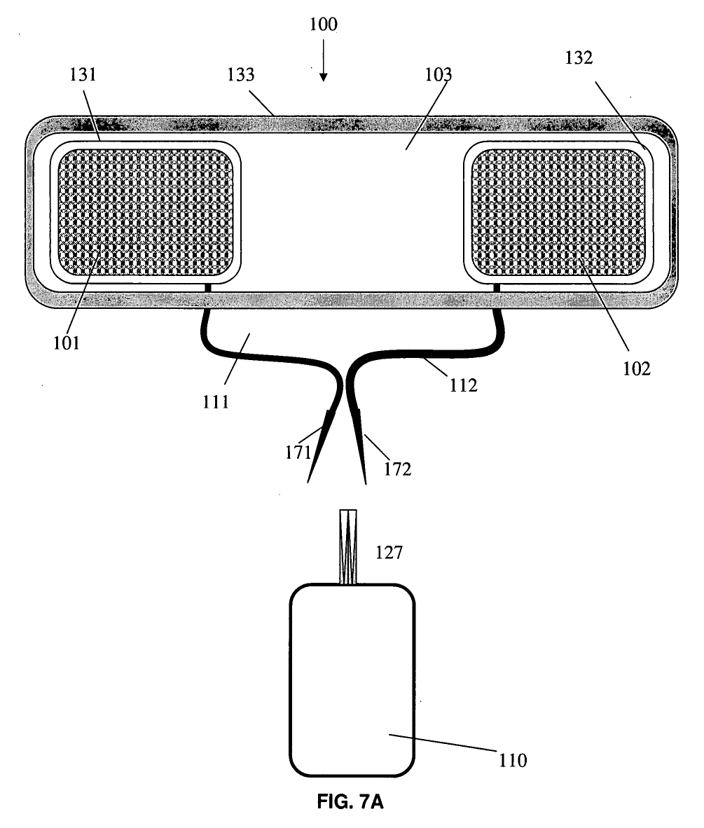

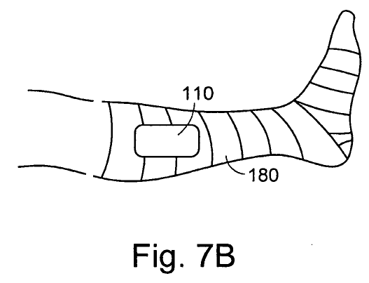

With reference to FIG. 7A, an apparatus 100 for treating an area of injured tissue according to a second embodiment of the invention comprises two electrodes 101 and 102 formed from carbon fibre woven cable and provided on a soft fabric pad 103, electrical connectors 111 and 112 for supplying electrical signals to the electrodes 101 and 102, and an electrotherapy device 110 similar to electrotherapy device 10 of the previous embodiment, for delivering electrotherapy signals. The two electrode connectors 111 and 112 are also made of carbon fibre woven cable. Examples of such electrodes and electrode connectors are described in PCT application No. PCT/GB2007/000317 (published as WO 2007/088348) entitled “Wound Dressing”, in the name of Wound Solutions Ltd, incorporated in its entirety herewith by this specific reference thereto. The ends 171 and 172 of each connector 111 and 112, respectively, which are to be connected to the electrical generator 110, are shaped in a piercing form such that they can penetrate through at least four layers of compression dressing for connection to the electrical generator 110 located outside the dressing. Electrode port 127 of electrical generator 110 is configured to receive the piercing ends 171 and 172. An example of an electrical connector having a piercing shaped end is described in PCT application PCT/GB2007/001842 (published as WO 2008/003920) entitled “Method and apparatus for monitoring external physical parameters having an influence on the onset or progression of a medical condition”, to Wound Solutions Ltd., incorporated in its entirety herewith by this specific reference thereto. Electrically conductive gel is spread on the exposed surface of the electrodes 101 and 102 to electrically couple and releasably adhere the electrodes to the skin of a patient during treatment. Strips of electrically insulating gel 131 and 132 are placed around the electrodes 101 and 102 respectively to prevent the ingress of moisture to the electrodes and the connectors at the connection to the electrodes. The outer edges of the pad 103 contain adhesive material 133 for adhering the pad 103 to the skin so that electrodes 101 and 102 are in contact with the skin of the patient.

It will be appreciated that the electrodes and the electrode leads may be made of any flexible conductive material or yarn. For example the electrode lead may be made up of a conductive filament intertwined in soft material.

Such an arrangement is extremely advantageous for patients receiving electrotherapy for the treatment of injured tissue such as a venous leg ulcer. The skin of these patients is extremely fragile and vulnerable to the formation of wounds and ulcers when pressed against hard objects. Since the connector feeding signals to the electrodes must be rooted to the area of treatment and in many cases the patient may be wearing a compression dressing, the compression dressing presses the cable against the skin of the patient. These may lead to the skin being broken and sores developing. The fabric cable helps to prevent this occurring since it does not result in localised pressure against the leg of the patient. Similarly the fabric electrodes placed on the delicate skin surrounding the injured tissue during treatment help to prevent further damage to the skin.

Accordingly, the method of one embodiment is particularly suited for use in conjunction with conventional methods of treatment of venous leg ulcers. The pad 103 is placed on the skin of the patient in the region of the venous leg ulcer with the electrodes arranged at each side of the ulcer. The leg is then fitted with a compression dressing in a similar manner as described for the previous embodiment. The electrical generator 110 can be clipped to the compression dressing 180 as shown in FIG. 7B.

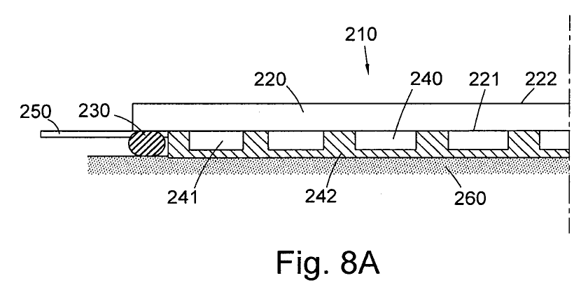

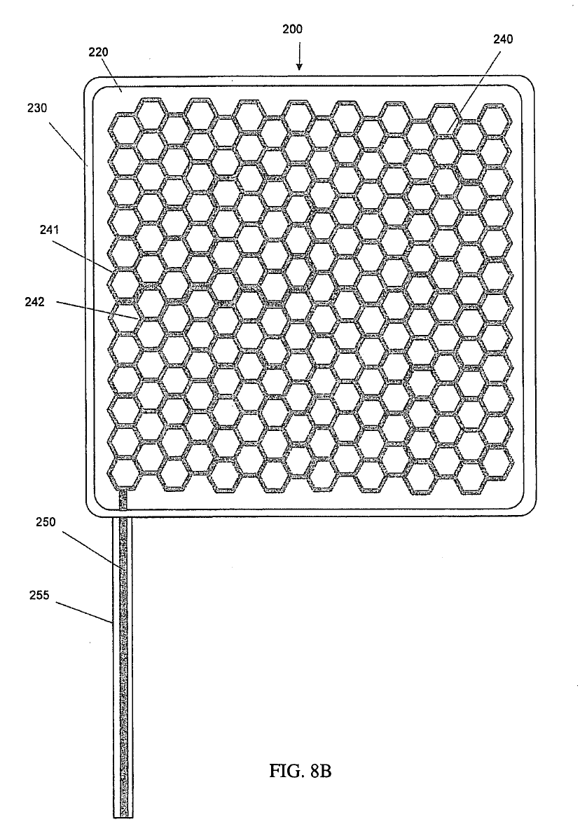

A third embodiment of an electrode arrangement for applying electrical signals to the region of treatment is illustrated in FIGS. 8A and 8B. An example of such an electrode arrangement is described in co-pending applications U.S. Ser. No. 11/138,358 (published as US2006173523) and U.S. Ser. No. 11/802,201 (published as ______) entitled “Electrode arrangement for applying electrical signals to the skin of an animal”, to Wound Solutions Ltd incorporated in their entirety herewith by this specific reference thereto. The electrode arrangement 210 comprises a flexible electrically non-conducting printed circuit board 220 with an extended portion 255 and having a first surface 221 and a second surface 222 opposed to the first surface, electrically non-conductive sealing gel 230 an electrode 240 for applying electrical signals to the skin 260 when placed in contact with the skin, and an electrically conductive connector 250 for supplying electrical signals to the electrode 240. The electrode 240 is formed from partial etching of a layer of electrically conductive material, such as gold plated copper, on the first surface 221 of the printed circuit board 220 and comprises electrically conductive tracks 241 with gaps there between. Electrically conductive gel 242 is placed in the gaps between the electrically conductive tracks 41 and over the surface of the electrically conductive tracks 241. The extended portion 255 of the printed circuit board 220 carries the flexible electrical connector 250 which when connected to an electrical current generator can carry current to the electrode 240. The portion of the flexible electrical connector on the extended portion is electrically insulated by an insulating layer 256 over the flexible electrical connector 250 to prevent any stray currents and short circuits. The extended portion 255 provides a flat lead to the electrode which will not indent the skin of a patient or cause discomfort and will not appear as a raised region or bulge when covered with a bandage or other type of medical dressing. It will be understood that the length of the extended portion is such that it extends from the wound area along the leg of the patient to provide a connection between the electrode 240 located in the area of the wound under a dressing and an electrical generator located outside the dressing.

The electrically non-conductive gel 230 is waterproof and is placed around the edges of the first surface 221 of the printed circuit board 220 to provide a seal between the electrode arrangement 210 and the skin 260 preventing ingress of moisture such as sweat, urine blood or wound exudate to the electrode 240 and ingress of moisture to any exposed part of the electrical connector 250 and the electrically conductive gel 242 which may lead to short circuits, stray currents and other undesirable effects. The electrode arrangement is sealed by the substrate 220, the insulating covering on the conductive lead 250, and the sealing gel 230 to prevent ingress of any fluid to the electrical path from the conductive lead 250 to the skin 260. The components of the electrode arrangement are washable so that they may be reused if required.

FIG. 8B is a schematic plan view of the electrode arrangement 210 showing the first surface 221 of the printed circuit board 220. The electrically conductive tracks 241 are arranged in the form of a honeycomb mesh structure with the electrical connector 250 formed on an extended portion 255 of the printed circuit board 220. The electrically conductive gel 242 is spread over the first surface 221 of the printed circuit board 220 into the gaps between the electrically conductive tracks 241 thereby forming further electrically conductive connections between the electrically conductive tracks 241, enhancing electrical conductivity across the surface of the electrode and providing a electrically conductive path between the electrode 240 and the skin. The sealing gel 230 is placed on a non-conductive portion 223 at the edges of the first surface 221 of the printed circuit board 220 in the form of a strip around the periphery of the printed circuit board 220. The printed circuit board 220 is fabricated from any suitable substrate such as a flexible polyester film. The combination of the honeycomb mesh pattern of electrically conductive tracks 241 and electrically conductive gel 242, with the polyester film printed circuit board provides an electrode arrangement which is flexible in a direction perpendicular to the plane of the electrode arrangement. This flexibility allows the electrodes 240 to make good electrical contact with the uneven or curvaceous surface of the skin when placed on the skin 260 with the first surface 221 of the printed circuit board 220 placed facing the skin 260. Furthermore this arrangement helps to eliminate hot spots resulting from uneven electrical-skin contact.

It should be appreciated that the gaps between the electrically conductive tracks 241 need only be sufficiently large to permit flexion of the printed circuit board.

Alternative electrode arrangements such as that described in co-pending applications U.S. Ser. No. 11/138,358 (published as US2006173523) and U.S. Ser. No. 11/802,201 (published as ______) may be used to provide the required flexibility. Alternative sealing arrangements such as described in co-pending applications U.S. Ser. No. 11/138,358 (published as US2006173523) and U.S. Ser. No. 11/802,201 (published as ______) may be used to prevent ingress of moisture to the electrode and to exposed portions of the electrode connector.

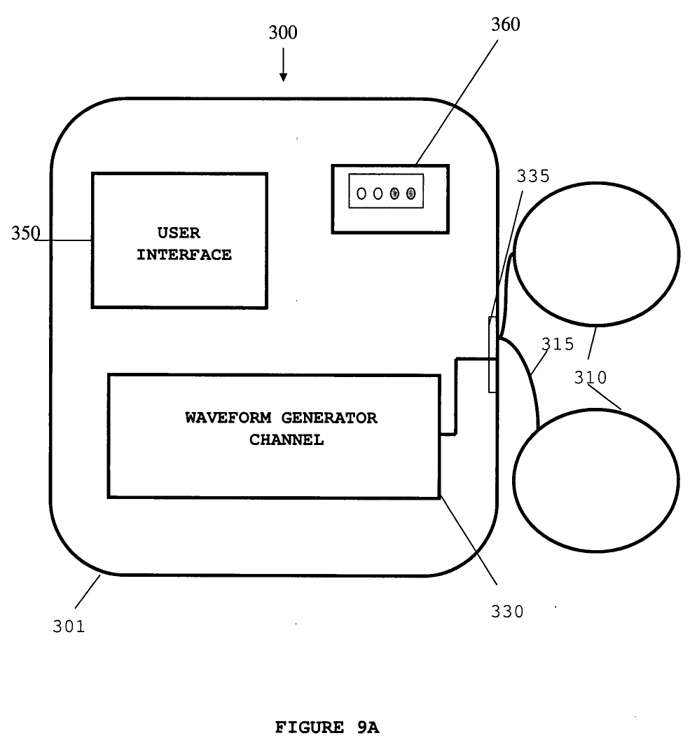

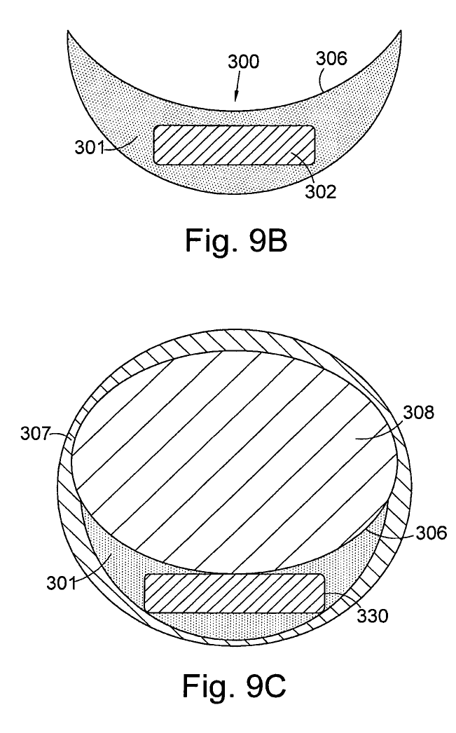

A fourth embodiment of an apparatus for treating a wound is described with reference to FIG. 9A. Apparatus 300 includes a waveform generator channel 330, similar to the waveform generator channel 40 of the first embodiment, housed in housing 301, an electrode port 335 for connecting to a pair of electrodes 310 via connectors 315, an LED status display 360 and a user interface 350.

Housing 301 will be described in more detail with reference to FIGS. 9B and 9C. Housing 301 is made of compressible foam and has a side wall 306 which is contoured to fit, in use, around part of the limb of a user. Under the influence of a force applied by a compression dressing 307 applied around the leg and the treatment apparatus 300 the housing 301 fits against the limb 308 of a user as illustrated in FIG. 9C. An example of such a compressible housing is described in co-pending PCT application PCT/GB2007/001842 (published as WO 2008/003920) incorporated herein by this specific reference thereto.

This device has the advantage that it provides a comfortable fit under a compression bandage and does not induce additional pain or cause secondary wounds. Further, the device does not impede the mobility of the wearer or interfere in their sleeping patterns

Status display 360 is configured such that the display is visible through up to four layers of a standard compression dressing. Thus, the status of device 300 can be ascertained without removing the compression dressing.

Waveform generator channel 330 is hermetically sealed against the ingress of moisture, electrodes 310 are sealed against the ingress of moisture to exposed electrically conductive parts in a similar manner to the electrodes of the second and third embodiments, and exposed electrically conductive parts of electrode connectors 315 are electrically insulated against the ingress of moisture. The apparatus is thereby sealed against the ingress of moisture when submitted to compression, for example, a pressure of up to 50-60 mmHg for up to 7-9 days.

Although in this embodiment the housing 301 is made of a foam-like material, in alternative embodiments the housing 301 may be made of any compressible elastomeric material.

In yet further embodiments the compression dressing may form part of the apparatus for treating injured tissue with the housing 301 being fitted in the compression dressing.

Referring to FIG. 10 an apparatus 400 for treating a wound according to a fifth embodiment of the invention comprises a waveform generator channel 430, an electrode port 435 for relaying signals from the waveform generator channel 430 to a pair of electrodes 410 via electrical connectors 415, a display 460, a user interface 450, a pressure sensor 405, a tilt sensor 406, a memory 407 and a data interface 408 through which data from the memory 407 can be downloaded.

Pressure sensor 405 is configured to measure pressure applied to soft tissue on the limb of a user by a compression dressing. The pressure sensor 405 may be a semiconductor, resistive or any proprietary small pressure measuring transducer.

For example, the pressure sensor may be a pressure sensor made of Quantum tunneling composite (QTC), a pressure sensor made up of a series of membrane switches that are designed to operate at different pressures by varying the material of the membrane or aperture in the spacer between the top and bottom contacts, a pneumatic sensor using a sealed partially inflated “sausage shaped balloon” to which is attached a single pressure sensor to take the average pressure of a compression bandage over that length or any other suitable pressure transducer known in the art.

In order to ensure that the pressure sensor 405 does not record pressure at a single point the pressure sensor 405 is mounted on a substrate to spread the loading pressure over as large an area as possible. In this way the pressure sensor can average the pressure over a significant area.

In alternative embodiments of the invention the pressure sensor 405 may be made up of multiple small pressure sensors spread over an area of treatment. This would enable the measurement of a whether a suitable relative pressure gradient is achieved or not. The multiple pressure sensors may be provided on one strip or alternatively they may be separate to one other and configured to communicate wirelessly to provide a pressure gradient.

An example of such a suitable pressure sensor is described in UK patent application GB0701129.9 (published as ______) entitled a flexible pressure sensor, in the name of Wound Solutions Ltd. GB0701129.9 (published as ______) is incorporated herein in its entirety by this specific reference thereto.

Waveform generator channel 430 operates in a similar manner to waveform generator channel 30 of the first embodiment.

The tilt sensor 406 comprises an inclinometer configured to measure the angle of the limb of a person relative to the direction of gravitational force i.e. relative to a line of gravity, when the treatment apparatus 400 is fitted on the limb of the user. Such devices are well known in the art and include devices based on the movement of conductive fluid, a pendulum or contacting elements. An example of a suitable tilt sensor is described in PCT application PCT/GB2007/01842 (published as WO 2008/003920) entitled “Method and apparatus for monitoring external physical parameters having an influence on the onset or progression of a medical condition, in the name of Wound Solutions Ltd and incorporated herein by this specific reference thereto. The tilt sensor 406 can also detect motion as well as or instead of tilt.

Measurements of the pressure recorded by pressure sensor 405, tilt angle measured by tilt sensor 406 and waveforms generated by waveform generator channel are recorded in memory 407 of the device 400. Data can be downloaded via interface 408 for analysis of the treatment regime applied over a certain period of time. A healthcare provider can thus see if the correct waveform was generated, the correct pressure was being applied and the leg was correctly tilted during certain periods.

Display 460 is configured to display the status of treatment apparatus 400 and data representative of measurements made by the tilt sensor 406 and the pressure sensor 405. For example the display may indicate elevation when an acceptable angle of tilt has been achieved or indicate the angle at which the limb is held, indicate when an appropriate pressure is being applied by the compression dressing or give a reading of the pressure measured by the pressure sensor.

An elevation may be typically defined as around 15 degrees above the horizontal, the horizontal being perpendicular to the direction of gravitational force. In the case of monitoring the angle of leg tilt for the treatment of venous leg ulcers the recommended angle of tilt would be in the range of from 0 degrees to the horizontal to 30 degrees above the horizontal.

In the case of an ambulatory unit, the inclinometer results may further be used to determine the appropriate time to begin an electrotherapy cycle and/or to select a modified form of electrotherapy cycle.

The indication of tilt helps to encourage the patient to keep the leg elevated when he or she is not mobile. Elevating the lower leg above the hip level aids blood flow return to the heart. Ideal elevation comprises bringing the foot above the level of the heart as this action helps to drain the lower leg with the aid of gravity while bringing the leg to a horizontal position helps to reduce venous pressure build-up or the formation of blood clots.

The indication of pressure helps to inform the patient if the correct pressure is being applied to the area of treatment or to alert the patient that an incorrect pressure caused for example by loosening of the compression dressing is being applied.

Indicating that the correct pressure is being applied to the wound area allows medical personnel applying a compression bandaging to ensure that the compression bandaging has been applied correctly. Applying compression bandaging correctly so as to achieve sustained compression for the duration of the treatment requires a high degree of skill, particularly with multi-layer bandaging. Achieving the required consistent gradients of pressures can be difficult and can lead to poorly applied bandages that can easily loosen thereby becoming ineffective. Furthermore, the patient can check that the correct pressure is being maintained throughout the duration of the treatment.

Further examples of monitoring the treatment regime are described in international patent application PCT/GB2007/01842 (published as WO 2008/003920) incorporated herein by reference thereto.

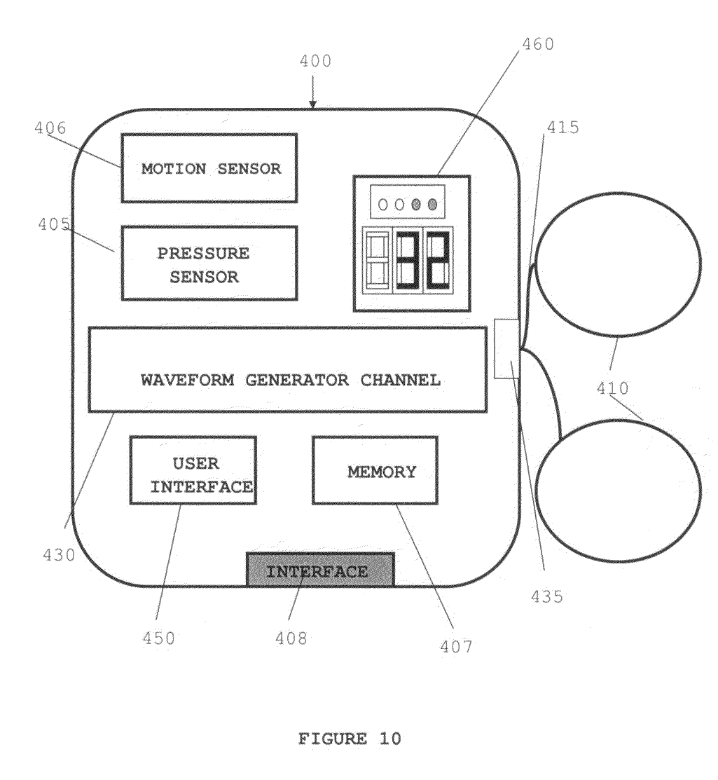

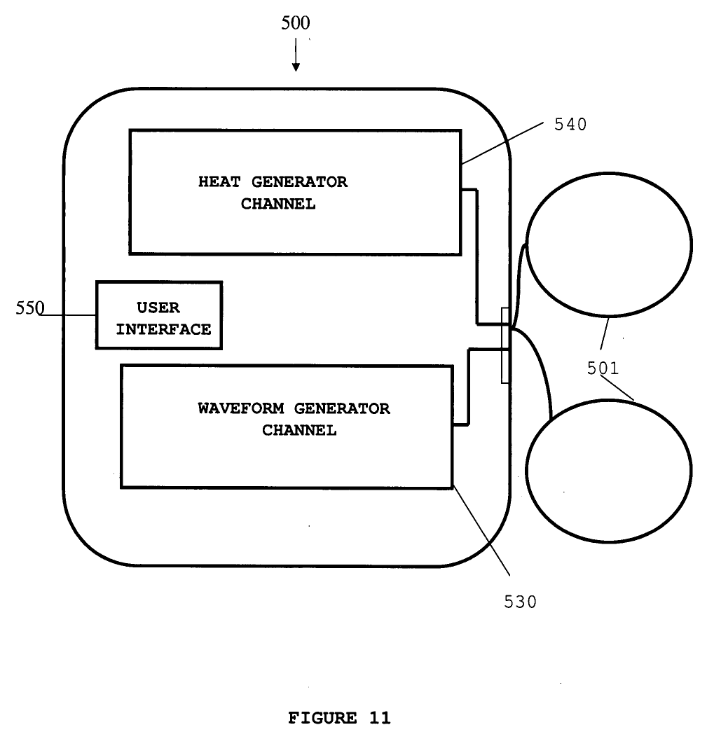

Referring to FIG. 11 a wound treatment device 500 according to a sixth embodiment of the invention comprises a pair of treatment pads 501, an electrical waveform generator 530, a treatment delivery device 540 and a user interface 550.

Electrical waveform generator channel 530 functions in a similar manner to electrical waveform generator channel 30 of the first embodiment and is arranged to deliver electrical signals to treatment pads 501. Treatment delivery device 540 is arranged to deliver heat energy to treatment pads 501 so that heat therapy can be applied to a treatment area in conjunction or as an alternative to electrotherapy. A compression dressing is placed over the area of treatment to provide a further form of therapy in addition to electrotherapy and heat therapy.

User interface 550 can be used to select the required treatment regime, for example electrotherapy, heat therapy or electrotherapy and heat therapy. Alternatively the device may be programmed to deliver a predetermined treatment regime and user interface 550 may be used to start and stop the treatment regime.

In further embodiments of the invention as an alternative or in addition to heat energy other therapeutic forms of energy may be applied to the area of the wound. Such forms of energy may include one or more of the following: electromagnetic, magnetic, mechanical or ultrasonic energy.

It will be appreciated that in alternative embodiments of the invention the additional form of therapy may be delivered by one or more treatment pads provided in addition to the electrode pads delivering electrotherapy to the area of treatment.

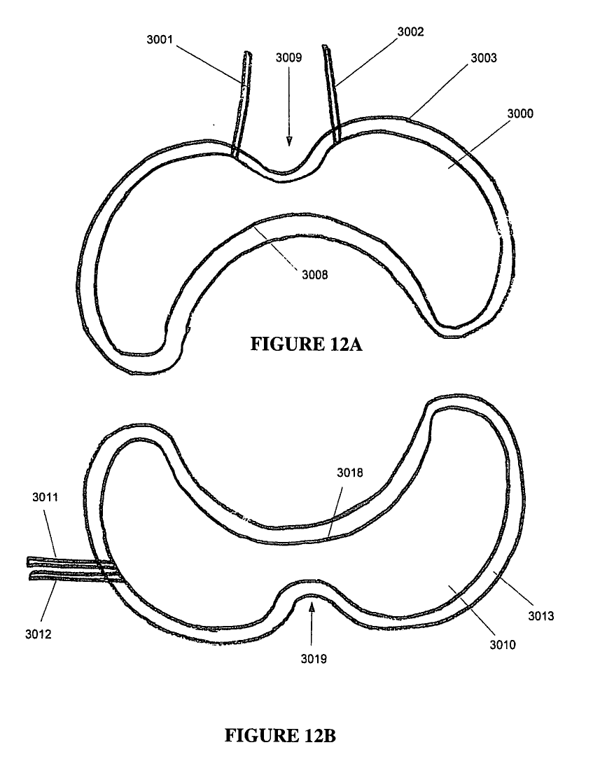

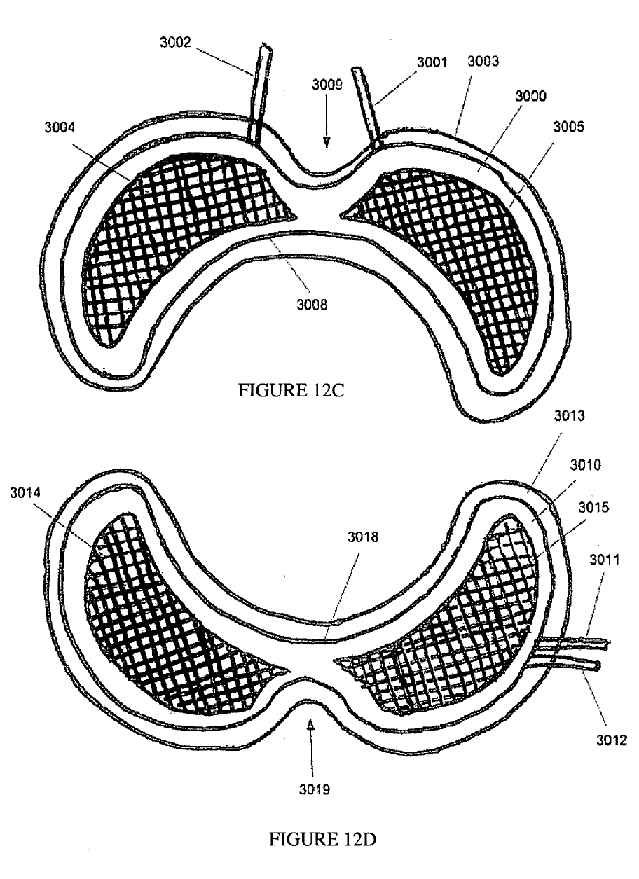

FIGS. 12A and 12C are diagrams illustrating electrode designs for electrodes of a treatment device according to a seventh embodiment of the present invention. In the design the electrode is shaped as two linked crescent shaped electrode regions lying in a generally part annular shape. The generally crescent shaped regions are linked at their apices. The electrode is formed of a non-conductive substrate 3000 which can be waterproof to provide a sealed electrode. The substrate 3000 is shaped as two crescent shaped portions linked at their ends to form a part annular shape having a concave circumferential inner edge 3008 and a circumferential outer edge shaped to form a recess 3009. On a first surface (generally a ‘bottom’ surface which will lie towards the skin of the patient) of each crescent shaped region of the substrate 3000 electrically conductive electrode regions 3004 and 3005 are provided. The electrode regions 3004 and 3005 are formed as layers of conductive material on the substrate 3000 in a similar manner to previous embodiments. The substrate 3000 and electrode regions 3004 and 3005 can comprise a flexible printed circuit board. The electrode regions 3004 and 3005 can be formed as a mesh or matrix of conductive material, a solid layer of conductive material, or a combination of a lower conductivity solid layer plus a layer of higher conductivity mesh or a matrix on either side of the solid lower conductivity layer. On a first surface of each electrode region 3004 and 3005 lying adjacent to the substrate 3000, respective electrical connector leads 3001 and 3002 electrically connect to the electrode regions 3004 and 3005 and lie between the substrate 3000 and the electrode regions 3004 and 3005.

The substrate 3000 extends beyond the electrode regions to form a flange region which is for adherence to the skin of the patient. Thus at least the flange regions of the substrate have an adhesive material applied thereto which is preferably waterproof to form a seal around the electrode regions 3004 and 3005 when the substrate 3000 is adhered to the skin of the patient. The substrate 3000 can also have an adhesive applied to the first surface in the region of the electrode regions 3004 and 3005 in order to bond the substrate to the electrode regions 3004 and 3005 and to bond the electrical connector leads 3001 and 3002 to the substrate 300 and the electrode regions 3004 and 3005 and to secure the electrical connection between the electrode regions 3004 and 3005 and the electrical connector leads 3001 and 3002.

The electrical regions 3004 and 3005 are provided with an electrically conductive gel on a second face away from the substrate for providing an electrical path to the skin of the patient from the electrode regions 3004 and 3005. The gel can be adhesive in nature to assist in the adherence of the electrode to the skin of the patient.

A release layer 3003 is provided across the electrode on the adhesive flange region of the substrate 3000 and on the adhesive second side of the electrode regions 3004 and 3005 to allow for storage of the electrode. The release layer 3003 is removed in order to adhere the electrode to the skin of the patient.

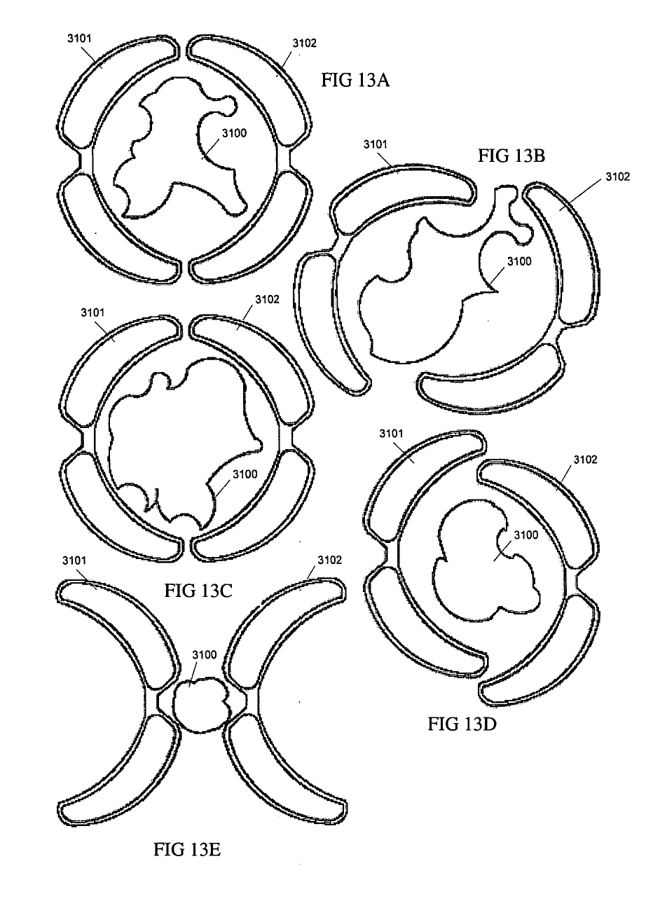

In this embodiment the electrical connector leads 3001 and 3002 are illustrated as being connected to and leaving the substrate in parallel either side of the recess in the outer circumferential edge of the substrate. While this provides for a symmetrical structure, the electrical connector leads 3001 and 3002 can get in the way of application of two of the electrodes around a small wound as will be described with reference to FIG. 13E.

FIGS. 12B and 12D illustrate the top and bottom views of an electrode according to an eighth embodiment of the present invention. This design of electrode is identical to the design of the seventh embodiment of the invention except for the placement of the electrical connector leads 3011 and 3012. In this design the leads 3011 and 3012 exit from the substrate 3010 at a region remote from the recess 3019. This ensures that the leads 3011 and 3012 will not lie in the region of a small wound as illustrated in FIG. 13E. In order for the leads of a pair of electrodes placed either side of a wound to lie in parallel, a pair of electrodes can be provided having leads exiting from the substrate 3010 at opposite crescent shaped regions ie. they can be mirror image designs.

FIGS. 13A to 13E illustrate placement of the electrodes of the seventh or eighth embodiments of the invention around different size and shapes of wounds. In FIGS. 13A to D it can be seen that the electrodes 3101 and 3102 are arranged with their inner circumferential edges facing the wound 3100. They can be arranged in slightly different positions to get close to the wound 3100. In FIG. 13E it can be seen that the wound 3100 is small and hence the inner circumferential edge of the electrodes is too large a diameter to enable the electrodes to be positioned close to the wound. Hence the electrodes are placed with the recessed portion of their outer circumferential edge adjacent to the wound. In this way the electrode regions are in close proximity to the wound to provide electrical stimulation therapy for wound healing.

It can thus be seen that the joined crescent shaped electrodes enable closer placement of the electrode regions to wounds with a variety of shapes and sizes. Current can be passed between any of the four electrode regions or between diagonally opposed pairs only.

The leads in this embodiment of the invention can be formed as soft leads in the same manner as the second embodiment of the invention or flat leads in the same manner as the third embodiment of the invention, thus making the electrode suitable for use under a compression dressing.

Examples of such an electrode design is described in U.S. application Ser. No. 11/802,201 (published as ______) incorporated herein by the specific reference thereto.

In further embodiments of the invention, a plurality of pairs of electrodes may be placed around the wound. The inner edge of the electrode pads may be placed at different distances from the outer edges of the wound. Although in some of the embodiments described above the electrode pads extend beyond the area of the wound it will be appreciated that in alternative embodiments the electrodes may not extend beyond the area of the wound. In alternative embodiments the electrodes may be placed in the wound.

In some embodiments of the invention three or more electrodes may be placed around the wound in a manner such as described in U.S. applications U.S. Ser. No. 11/138,358 (published as US2006173523A1) and U.S. Ser. No. 11/802,201 (published as ______). Such a configuration allows electrical current to pass through different paths through the tissue under the wound thereby promoting healing of the injured tissue.

Although in the above-described embodiments compression bandaging is used as a compression covering, in alternative embodiments other types of compression coverings applying controlled pressure to veins to improve blood flow in the region of a wound, such as compression hosiery may be used.

In alternative embodiments of the invention, the waveform applied during the first treatment stage comprises a series of current pulses having an amplitude in a range of from 80 to 300 μA, having a frequency in a range from 0.5 to 1.5 pulses per second and a pulse width in a range from 333 to 1000 ms, the waveform applied during the second stage of treatment comprises a series of current pulses having an amplitude in a range of from 20 to 60 μA, having a frequency in a range from 2 to 4 pulses per second and a pulse width in a range from 125 to 250 ms, and the waveform applied during the third stage of treatment comprises a series of current pulses having an amplitude in a range of from 250 to 640 μA, having a frequency in a range of from 80 to 120 pulses per second and a pulse width in a range from 4 to 6 ms.

In alternative embodiments of the invention the polarity of the electrodes may be reversed at periodic intervals of approximately 5 to 15 seconds. In further embodiments the polarity of the electrodes may not be reversed.

While the treatment stages may last for longer or shorter periods, in another embodiment of the invention the first treatment stage lasts for a period of time ranging from 4 to 6 minutes, the second treatment stage lasts for a period of time ranging from 25 to 50 minutes, the third treatment stage lasts for a period of time ranging from 20 to 35 minutes. In a further embodiment of the invention, each treatment stage lasts for a period of time ranging from 5 to 40 minutes.

Although in the embodiment described hereinabove, the electrodes are placed either side of the wound, in one aspect the present invention is not so limited and electrodes could be placed at any angle to the wound e.g. in a wound dressing, when used with the waveforms described herein and compression coverings to provide improved wound healing.

Further, although the embodiment described hereinabove, a compression covering is used, in one aspect the present invention is not so limited. An improved wound treatment method can be achieved using electrodes either side of the wound and the waveforms described herein.

Further features of electrode arrangements such as those described in U.S. Ser. No. 11/138,358 (published as US2006173523A1) and PCT application PCT/GB2006/00315 (published as WO2006082384) may be used for the application of electrical signals to the skin of a person. The entire contents of these patent applications is incorporated in their entirety by this specific reference thereto.

Although the present invention has been described with reference to specific embodiments, it will be apparent to a skilled person in the art that modifications lie within the spirit and scope of the present invention.

What is claimed is:

1. A method of treating injured tissue or preventing injury to tissue susceptible to injury, the method comprising:
positioning a plurality of electrodes spaced apart in the region of said tissue; placing a compression covering over the plurality of electrodes and the region of said tissue to apply pressure in the region of said tissue; and
applying a sequence of electrical waveforms between at least two of the plurality of electrodes, the sequence of electrical waveforms comprising:
a first waveform comprising a series of current pulses having an amplitude in a range of from 80 to 300 μA, having a frequency in a range from 0.5 to 1.5 pulses per second and a pulse width in a range from 333 to 1000 ms;
a second waveform comprising a series of current pulses having an amplitude in a range of from 20 to 60 μA, having a frequency in a range from 2 to 4 pulses per second and a pulse width in a range from 125 to 250 ms; and
a third waveform comprising a series of current pulses having an amplitude in a range of from 250 to 640 μA, having a frequency in a range of from 80 to 120 pulses per second and a pulse width in a range from 4 to 6 ms.

2. A method according to claim 1, wherein at least one of the first waveform, the second waveform and the third waveform comprises a first part comprising said pulses of a first polarity and a second part comprising pulses of a second polarity.

3. A method according to claim 2, wherein during application of at least one of the first waveform, the second waveform and the third waveform the polarity of the electrodes is reversed approximately every 5 to 15 seconds.

4. A method according to claim 3, wherein during application of at least one of the first waveform, the second waveform and the third waveform the polarity of the electrodes is reversed at substantially every 10 seconds.

5. A method according to claim 1, wherein
the first waveform comprises a series of current pulses having an amplitude of substantially 100 μA, a frequency of substantially 1 pulse per second and a pulse width of substantially 500 ms;
the second waveform comprises a series of current pulses having a amplitude of substantially 40 μA, a frequency of substantially 3 pulses per second and a pulse width of substantially 166 ms;
the third waveform comprises a series of current pulses having an amplitude of substantially 320 μA, a frequency of substantially 100 pulses per second and a pulse width of substantially 5 ms.

6. A method according to claim 1, wherein the electrodes are positioned in contact with skin peripheral to said tissue.

7. A method according to claim 6, wherein each electrode of a pair of electrodes is positioned on opposite sides of said tissue to one another.

8. A method according to claim 6, wherein each electrode is placed approximately 1 cm from an outer boundary of the area of said tissue.

9. A method according to claim 1, wherein the first waveform is generated over a period of time ranging from 2 to 8 minutes.

10. A method according to claim 9, wherein the first waveform is generated over a time period of substantially 5 minutes

11. A method according to claim 1, wherein the second waveform is generated over a period of time ranging from 25 to 45 minutes.

12. A method according to claim 11, wherein the second waveform is generated over a period of time of substantially 35 minutes.

13. A method according to claim 1, wherein the third waveform is generated over a period of time ranging from 20 to 35 minutes.

14. A method according to claim 13 wherein the third waveform is generated over a period of time of substantially 25 minutes.

15. A method according to claim 1, wherein the first waveform is generated over a period of time ranging from 5 to 10 minutes, the second waveform is generated over a period of time ranging from 25 to 50 minutes, and the third waveform is generated over a period of time ranging from 15 to 30 minutes.

16. A method according to claim 15, wherein the first waveform is generated over a period of time of substantially 5 minutes, the second waveform is generated over a period of time of substantially 35 minutes, and the third waveform is generated over a period of time of substantially 25 minutes.

17. A method according to claim 1, wherein the pulses are substantially rectangular.

18. A method according to claim 1, wherein the sequence of waveforms is repeated.

19. A method according to claim 1, further comprising applying therapeutic energy to the region of said tissue.

20. A method according to claim 19, wherein applying therapeutic energy to the region of said tissue comprises applying at least one of vibratory, chemical, electromagnetic, heat, mechanical, ultrasonic, and magnetic energy to the region of injured tissue.

21. A method of treating injured tissue or preventing injury to tissue susceptible to injury, comprising:
placing a plurality of electrodes on the skin in an area of said tissue;
placing a compression covering over the electrodes and the area of said tissue to blood flow in the area of said tissue; and
applying an electrical current to pass from at least one electrode to at least one other electrode of the plurality of electrodes.

22. A method according to claim 21 wherein the electrodes are positioned on the skin on opposite sides of the area of said tissue to one another.

23. A method according to claim 22, wherein each electrode is placed approximately 1 cm from an edge of the area of said tissue.

24. A method according to claim 21, wherein opposing ends of each electrode extend beyond the outermost edges of the area of said tissue.

25. A method according to claim 24, wherein opposing ends of each electrode extend beyond the outermost edges of the area of said tissue by approximately 1 to 1.5 cm.

26. A method according to claim 25, further comprising applying therapeutic energy to the area of said tissue.

27. A method according to claim 26, wherein applying therapeutic energy to the area of said tissue comprises applying at least one of electromagnetic, heat, mechanical, ultrasonic, and magnetic energy to the area of said tissue.

28. A method according to claim 21, wherein at least three electrodes are placed in contact with skin in a region peripheral to the area of said tissue, the method further comprising switching the electrical current to flow between different electrodes of the at least three electrodes.

29. A method according to claim 21, further comprising at least one of measuring the pressure applied by the compression covering to the area of said tissue and measuring the angle of tilt at which a limb having the said tissue is held or moved with respect to the direction of gravitational force.

30. A method according to claim 21, wherein one or more of the electrodes is each provided on a flat substrate shaped as two substantially crescent shaped portions apically linked and having a concave inner circumferential edge and a convex outer circumferential edge, said outer circumferential edge having an indented region situated at the apically linked region between said substantially crescent shaped portions and the method includes placing each substrate close to the periphery of the injured tissue by selecting to place either the inner circumferential edges of said plurality of said electrodes adjacent to the said tissue, or the indented region of the outer circumferential edges of said plurality of said electrodes adjacent to the said tissue.

31. A method according to claim 21, wherein said electrodes are placed in contact with skin in a region peripheral to the area of said tissue.

32. A method of treating a wound, comprising:
placing a plurality of electrodes in contact with skin in a region peripheral to the wound; and
applying a sequence of current waveforms between the electrodes, the sequence of current waveforms comprising:
a first waveform comprising a series of current pulses having an amplitude in a range of from 80 to 300 μA, having a frequency in a range from 0.5 to 1.5 pulses per second and a pulse width in a range from 333 to 1000 ms;
a second waveform comprising a series of current pulses having an amplitude in a range of from 20 to 60 μA, having a frequency in a range from 2 to 4 pulses per second and a pulse width in a range from 125 to 250 ms; and
a third waveform comprising a series of current pulses having an amplitude in a range of from 250 to 640 μA, having a frequency in a range of from 80 to 120 pulses per second and a pulse width in a range from 4 to 6 ms.

33. A method according to claim 32, wherein at least one of the first waveform, the second waveform and the third waveform comprises a first part comprising said pulses of a first polarity and a second part comprising pulses of a second opposite polarity.

34. A method according to claim 33, wherein during application of the at least one of the first waveform, the second waveform and the third waveform the polarity of the electrodes is reversed approximately every 5 to 15 seconds.

35. A method according to claim 34, wherein during application of at least one of the first waveform, the second waveform and the third waveform the polarity of the electrodes is reversed at substantially every 10 seconds.

36. A method according to claim 32, wherein:
the first waveform comprises a series of current pulses having an amplitude of substantially 100 μA, a frequency of substantially 1 pulse per second and a pulse width of substantially 500 ms;
the second waveform comprises a series of current pulses having a amplitude of substantially 40 μA, a frequency of substantially 3 pulses per second and a pulse width of substantially 166 ms;
the third waveform comprises a series of current pulses having an amplitude of substantially 320 μA, a frequency of substantially 100 pulses per second and a pulse width of substantially 5 ms.

37. A method according to claim 32, wherein each electrode of a pair of electrodes are positioned on opposite sides of the wound to one another.

38. A method according to claim 37, wherein each electrode is placed approximately 1 cm from an edge of the wound.

39. A method according to claim 32, wherein the first waveform is generated over a period of time ranging from 2 to 8 minutes.

40. A method according to claim 39, wherein the first waveform is generated over a time period of substantially 5 minutes

41. A method according to claim 32, wherein the second waveform is generated over a period of time ranging from 25 to 40 minutes.

42. A method according to claim 41, wherein the second waveform is generated over a period of time of substantially 35 minutes.

43. A method according to claim 32, wherein the third waveform is generated over a period of time ranging from 15 to 35 minutes.

44. A method according to claim 43, wherein the second waveform is generated over a period of time of substantially 25 minutes.

45. A method according to claim 32, wherein the first waveform is generated over a period of time ranging from 2 to 8 minutes, the second waveform is generated over a period of time ranging from 25 to 40 minutes, and the third waveform is generated over a period of time ranging from 15 to 35 minutes.

46. A method according to claim 45, wherein the first waveform is generated over a period of time of substantially 5 minutes, the second waveform is generated over a period of time of substantially 35 minutes, and the third waveform is generated over a period of time of substantially 25 minutes.

47. A method according to claim 32, wherein the pulses are substantially rectangular.

48. A method according to claim 32, wherein the sequence of waveforms is repeated.

49. A method according to claim 32, further comprising applying therapeutic energy to the region of the wound.

50. A method according to claim 49, wherein applying therapeutic energy to the region of the wound comprises applying at least one of chemical, vibratory, electromagnetic, heat, mechanical, ultra sonic, and magnetic energy to the region of the wound.

51. A method according to claim 32, wherein at least three electrodes are placed in contact with skin in a region peripheral to the region of the wound, the method further comprising switching the electrical current to flow between different electrodes of the at least three electrodes.

52. A method according to claim 32, further comprising measuring at least one of the pressure applied by the compression covering to the region of the wound and measuring the angle of tilt at which a limb having the wound is held or moved with respect to the direction of gravitational force.

53. An apparatus for treating injured tissue, the apparatus comprising:
a waveform generator adapted to generate:
a first waveform comprising a series of current pulses having an amplitude in a range of from 80 to 300 μA, having a frequency in a range from 0.5 to 1.5 pulses per second and a pulse width in a range from 333 to 100 ms;
a second waveform comprising a series of current pulses having an amplitude in a range of from 20 to 60 μA, having a frequency in a range from 2 to 4 pulses per second and a pulse width in a range from 125 to 250 ms; and
a third waveform comprising a series of current pulses having an amplitude in a range of from 250 to 640 μA, having a frequency in a range of from 80 to 120 pulses per second and a pulse width in a range from 4 to 6 ms; and
output connectors for connection to an electrode arrangement for applying the waveforms through regenerative tissue underneath the injured tissue.

54. An apparatus according to claim 53, wherein said waveform generator includes a switch arrangement for switching the polarity of the pulses.

55. An apparatus according to claim 53, wherein the waveform generator is pre-programmed with one or more programs for generating one of said waveforms or a pre-determined sequence of said waveforms.

56. An apparatus according to claim 55, further comprising a user interface for selecting one of said waveforms or a predetermined sequence of said waveforms.

57. An apparatus according to claim 53, further adapted to generate:
a first waveform comprising a series of current pulses having an amplitude in a range of from 80 to 300 μA, having a frequency in a range from 0.5 to 1.5 pulses per second and a pulse width in a range from 333 to 1000 ms;
a second waveform comprising a series of current pulses having an amplitude in a range of from 20 to 60 μA, having a frequency in a range from 2 to 4 pulses per second and a pulse width in a range from 125 to 250 ms; and
a third waveform comprising a series of current pulses having an amplitude in a range of from 250 to 640 μA, having a frequency in a range of from 80 to 120 pulses per second and a pulse width in a range from 4 to 6 ms;
the apparatus further comprising other output connectors for connection to another electrode arrangement for applying the waveforms across the wound.

58. A wound treatment arrangement for treating a wound in the skin of a patient, the wound treatment arrangement comprising:
a compression dressing arrangement applied to a limb of the patient and applying pressure thereto;
a plurality of electrodes for applying electrical signals to the skin of a patient wherein said electrodes are arranged in the area of said wound under said compression dressing arrangement; and
an electrical generator for generating electrical current to pass from one of the plurality of electrodes to another of the plurality of electrodes.

59. A wound treatment arrangement according to claim 58, wherein the electrical generator is attachable to the compression dressing arrangement.

60. A wound treatment arrangement according to claim 58, wherein the electrical generator is disposed in housing providing a low profile under the compression dressing arrangement.

61. A wound treatment arrangement according to claim 60, wherein the housing is operable to compress under the influence of pressure exerted by the compression dressing arrangement such that it fits around at least part of the limb of said patient.

62. A wound treatment arrangement according to claim 61, wherein the compressible housing is made of a flexible elastomeric material.

63. A wound treatment arrangement according to claim 61, wherein the compressible housing is made of foam material.

64. A wound treatment arrangement according to claim 60, wherein a side of the housing is contoured to fit around a least part of the limb.

65. A wound treatment arrangement according to claim 58, further comprising at least one of a tilt sensor for measuring the tilt of the limb relative to the direction of gravitational force and a pressure sensor for measuring the pressure applied by the compression dressing.

66. A wound treatment arrangement according to claim 58, further comprising a display operable to provide a reading visible through the compression dressing arrangement.

67. A wound treatment arrangement according to claim 58, further comprising a treatment device operable to apply a therapeutic form of energy to said limb.

68. A wound treatment arrangement according to claim 67, wherein the treatment device is operable to apply at least one of chemical, vibratory, heat, mechanical, ultrasound, electromagnetic and magnetic energy to a region of the wound.

69. A wound treatment arrangement according to claim 58, further comprising electrode connectors for relaying electrical signals from the electrical generator to the plurality of electrodes, wherein each of the electrical connectors has a first end adapted to pierce through the compression dressing arrangement for connection to the electrical generator.

70. A wound treatment arrangement according to claim 58, further comprising an electrically non-conductive sealing arrangement for preventing ingress of moisture to electrically conductive portions of the plurality of electrodes and the electrical generator.

71. A wound treatment arrangement according to claim 69, further comprising an electrically non-conductive sealing arrangement for preventing ingress of moisture to electrically conductive portions of the plurality of electrodes, the electrical generator and the electrode connectors.

72. A wound treatment arrangement according to claim 70, wherein said electrically non-conductive sealing arrangement is arranged to effectively seal under a pressure of up to 50-60 mmHg constant pressure for up to 7 to 9 days.

73. A wound treatment arrangement according to claim 58, wherein each electrode is provided on a flat substrate shaped as two substantially crescent shaped portions apically linked and having a concave inner circumferential edge and a convex outer circumferential edge, said outer circumferential edge having an indented region situated at the apically linked region between said substantially crescent shaped portions.

74. A wound treatment arrangement according to claim 58, wherein said electrodes are arranged on said skin adjacent said wound.

75. A kit of parts for treating a wound in the skin of a patient, the kit comprising
a compression dressing arrangement for application to the limb of the patient to apply pressure thereto;
a plurality of electrodes for applying electrical signals to the skin of the patient; and
an electrical generator for generating electrical current to pass from one of the plurality of electrodes to the other of the plurality of electrodes.

76. An apparatus for treating a wound, comprising:
a pair of electrodes; and
means to generate a waveform for applying across said pair of electrodes; wherein the means to generate a waveform is adapted to generate a sequence of waveforms comprising:
a first waveform comprising a series of current pulses having an amplitude in a range of from 80 to 300 μA, having a frequency in a range from 0.5 to 1.5 pulses per second and a pulse width in a range from 333 to 1000 ms;
a second waveform comprising a series of current pulses having an amplitude in a range of from 20 to 60 μA, having a frequency in a range from 2 to 4 pulses per second and a pulse width in a range from 125 to 250 ms; and
a third waveform comprising a series of current pulses having an amplitude in a range of from 250 to 640 μA, having a frequency in a range of from 80 to 120 pulses per second and a pulse width in a range from 4 to 6 ms.

77. An apparatus according to claim 76, further comprising polarity switching means.

78. An apparatus according to claim 76, further comprising user interface means for selecting one of said waveforms or a predetermined sequence of said waveforms.

79. An apparatus according to claim 76, wherein the apparatus is operable to detect a high resistance condition between the pair of electrodes indicating poor electrical contact between one or both electrodes and the skin of a patient.

80. A method of treating a medical condition on the surface of or under the surface of a region of tissue, the method comprising:
positioning a plurality of electrodes spaced apart in the region of tissue;
placing a compression covering over the plurality of electrodes and the region of said tissue to apply pressure in the region of said tissue; and
applying a sequence of electrical waveforms between at least two of the plurality of electrodes, the sequence of electrical waveforms comprising:
a first waveform comprising a series of current pulses having an amplitude in a range of from 80 to 300 μA, having a frequency in a range from 0.5 to 1.5 pulses per second and a pulse width in a range from 333 to 1000 ms;
a second waveform comprising a series of current pulses having an amplitude in a range of from 20 to 60 μA, having a frequency in a range from 2 to 4 pulses per second and a pulse width in a range from 125 to 250 ms; and
a third waveform comprising a series of current pulses having an amplitude in a range of from 250 to 640 μA, having a frequency in a range of from 80 to 120 pulses per second and a pulse width in a range from 4 to 6 ms.
